(19) United States
(12) Patent Application Publication
(10) Pub. No.: US 2025/0221654 A1
(43) Pub. Date: Jul. 10, 2025

(54) BIOBEHAVIORAL SENSING PLATFORM FOR CAPTURING INFANT STATES/HEALTH AND ENVIRONMENTAL INPUT VIA ECG, MOTION, AND AUDIO SIGNALS

(71) Applicant: The Board of Trustees of the University of Illinois, Urbana, IL (US)

(72) Inventors: Nancy L. McElwain, Urbana, IL (US); Mark A. Hasegawa-Johnson, Champaign, IL (US); Romit Roy Choudhury, Champaign, IL (US); Ashutosh Dhekne, Atlanta, GA (US); Bashima Islam, Stow, MA (US)

(21) Appl. No.: 19/017,115

(22) Filed: Jan. 10, 2025

Related U.S. Application Data
(60) Provisional application No. 63/619,519, filed on Jan. 10, 2024.

(51) Int. Cl. A61B 5/352; A61B 5/00; A61B 5/16; A61B 5/256

(52) U.S. Cl. CPC A61B 5/352; A61B 5/0006; A61B 5/165; A61B 5/256; A61B 5/4806; A61B 5/746; A61B 2503/06

(57) ABSTRACT

Various systems and methods are provided herein for performing health status detection. In one embodiment, ECG data, acceleration data, and audio data is received via a sensor unit. One or more parameters are extracted from the ECG data. An activity associated with the acceleration data is determined. At least one of an emotional state, a behavioral state, or an infant-caregiving interaction associated with the audio data is detected. One or more physiological and behavioral indicators are determined based on the one or more parameters, the activity, and the emotion state, behavioral state, or the infant-caregiver interaction. A health status corresponding to the physiological and behavioral indicators is outputted.

### CROSS REFERENCE TO RELATED APPLICATIONS

This application claims priority to and incorporates by reference U.S. provisional patent application No. 63/619,519, filed Jan. 10, 2024.

### STATEMENT OF GOVERNMENT SUPPORT

This invention was made with government support under DA050256 awarded by the National Institutes of Health. The government has certain rights in the invention.

### BACKGROUND

Advances in personal sensing, Internet of Medical Things, and digital health have rapidly accelerated over the past decade, including the use of wearable devices among adults, children and adolescents, and infants. Innovations in infant wearables, in particular, have predominantly focused on wireless skin-interfaced biosensor made of soft, flexible electronics that permit continuous monitoring of vital signs, including but not limited to heart rate, blood pressure, temperature, respiration, and blood oxygen saturation. Such sensors present notable benefits over more conventional wired systems, such as decreased iatrogenic effects (e.g., less damage to the infant's delicate skin) and increased mobility (e.g., the infant can be picked up and held by a parent or caregiver, i.e., Kangaroo care). Nonetheless, their development and testing have been largely restricted to neonatal and pediatric intensive care units (NICU, PICU), calling into question their utility and feasibility with respect to in-home monitoring under free-living conditions, in which infant's movements, location, and environment may continuously change. Further, existing systems monitoring multiple vital signs typically require placement of sensors on different parts of the body, which increases complexity of the set up for the caregiver as well as potential discomfort or restriction in movement for the infant, all of which may further decrease feasibility for home use. Undoubtedly, these sensor systems address an important clinical need-to monitor the infant's physical health via detection of changes in vital signs in ways that are more patient-friendly. Understandably, however, these systems do not include sensing modalities, such as audio, that would permit assessment of the infant's biobehavioral development or social environment.

Infant wearables designed for in-home use have predominantly focused on a signal modality: audio, physiology, or motion. Prior work assessing a combination of behavioral (via audio, video, or motion sensors) and physiological (i.e., ECG, electroencephalography (EEG)) signals among infant samples in the home environment is extremely rare, and in these cases, separate data collection platforms or devices have been used to collect different data streams. Such methods yield rich data, but limitations include the complexity of the sensor set-up for parents to implement on their own, concerns about the child tolerating multiple sensors for prolonged periods of time, and challenges and pitfalls of post-hoc signal synchronization. The device described herein is a wearable that focuses on simultaneously and continuously monitoring cardiac physiology, motion, and vocalizations of infants and young children. Given no existing device can simultaneously collect data from all three modalities, there is a need for a compact multimodal platform to capture biobehavioral development and mental health of infants and young children from daylong recordings in the home context. Further, automated detection of infant biobehavioral states, health status, and key aspects of the caregiving environment (e.g., caregivers' vocal responses to infant cues, household noise/chaos) via machine learning algorithms trained on these multimodal data (ECG, motion, audio) will substantially increase efficiency and effectiveness of early prevention/intervention programs.

### SUMMARY

In one aspect, the present disclosure can provide a wearable sensor device including a housing, an electrocardiogram (ECG) sensor, a motion sensor, an audio sensor, and a wireless communication transmitter. The housing can be sized to fit within a chest pocket of subject's garment and can be free of affixing features. The ECG sensor can include internal circuitry within the housing and can have a set of electrodes configured to directly contact the subject's chest when the housing is disposed in the subject's garment. The ECG sensor can further conduct electrical signals to internal circuitry. The motion sensor can be disposed within the housing and the audio sensor can be disposed at least partially within the housing. The device can further include a processor and a memory. The processor can be in communication with the ECG sensor, the motion sensor, and the audio sensor. The memory can be in communication with the processor and can have instructions stored thereon that cause the processor to receive ECG data, acceleration data, and audio data via the ECG sensor, the motion sensor, and the audio sensor. The data can be transmitted via the wireless communication transmitter.

In another aspect, the present disclosure can provide a system for analyzing and monitoring biobehavioral development of a child or infant. The system can include a wearable device, which can include a plurality of child biobehavioral development sensor, a wireless communication transmitter, and a power source. The power source can provide power to the wearable device for a period of time. The wearable device can further include a processor configured to read data from the plurality of child biobehavioral development sensors and transmit the data via the wireless communication transmitter. The system can further include a data analysis system remote from the wearable device. The data analysis system can include a memory having instructions stored thereon that, when executed, cause the data analysis system to perform one or more steps. Data obtained from the plurality of child biobehavioral development sensors can be received from the wireless communication transmitter. An activity associated with the data can be determined. One or more physiological and behavioral indicators can be determined based on the data and the activity. A health status corresponding to the physiological and behavioral indicators can be outputted.

In another aspect, the present disclosure can provide a method for analyzing and long-term monitoring of child biobehavioral development. ECG data, acceleration data, and audio data can be received via a sensor unit. One or more parameters can be extracted from the ECG data. The one or more parameters can include a stress level. An activity associated with the acceleration data can be determined. At least one of an emotional state, a behavioral state, or an infant-caregiving interaction associated with the audio data can be detected. One or more physiological and behavioral indicators can be determined based on the one or more parameters, the activity, and the emotion state, behavioral state, or the infant-caregiver interaction. A health status corresponding to the physiological and behavioral indicators can be outputted.

### BRIEF DESCRIPTION OF THE DRAWINGS

The foregoing features of embodiments will be more readily understood by reference to the following detailed description, taken with reference to the accompanying drawings, in which:

FIG. 1 illustrates a block diagram conceptually illustrating a wearable infant monitoring system 100.

FIG. 2 is a flowchart illustrating an example process for performing health status detection according to some embodiments.

FIG. 3 illustrates an example flowchart for performing health status detection using a neural network model.

FIG. 4 illustrates an example of training a neural network or other suitable machine learning model to provide health status detection.

FIG. 5 is a block diagram of an example health monitoring system.

FIG. 6A illustrates a device enclosure according to some embodiments.

FIG. 6B illustrates ECG leads, electrodes, a charger, and a shirt according to some embodiments.

FIG. 6C illustrates an infant wearing a wearable monitor according to some embodiments.

FIGS. 7A-D illustrate Bland-Altman plots comparing interbeat intervals (IBI) extracted from wearable monitors for adult participants according to some embodiments.

FIG. 8 illustrates box plots depicting RSA task means, distributions, and outliers for adult participants using wearable monitors according to some embodiments.

FIGS. 9A-D illustrate Bland-Altman plots comparing interbeat intervals extracted from wearable monitors for infant participants according to some embodiments.

FIG. 10 illustrates box plots depicting RSA task means and distributions for infant participants using wearable monitors according to some embodiments.

FIG. 11 illustrates raw data from a wearable monitor and smartphone IMU sensors according to some embodiments.

FIG. 12 illustrates confusion matrices of activity classification with a wearable monitor and smartphone IMU data according to some embodiments.

FIG. 13 illustrates box plots showing performance of activity classification with a wearable monitor and smartphone IMU data according to some embodiments.

FIG. 14 illustrates confusion matrices of speech emotion recognition with a wearable monitor and smartphone audio data according to some embodiments.

FIG. 15 illustrates box plots showing performance of speech emotion recognition with a wearable monitor and smartphone audio data according to some embodiments.

### DETAILED DESCRIPTION

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Various embodiments, configurations, materials, devices, systems, methods, and techniques for performing health status detection are disclosed herein. With respect to the devices and systems described below, certain alternative components and materials are described, none of which are intended to be limiting or required. The description of components of such devices and systems is intended to be illustrative only, and neither a minimum nor limit of the types of components that could be used in various embodiments hereof. Similarly, the methods described herein are explained with reference to optional steps and modifications, none of which are intended to be limiting or required. The methods described herein can be performed using hardware such as (or including) the devices and systems described herein but need not be implemented through such hardware except in specific examples that identify the use of such hardware.

### Hardware Configurations and Embodiments

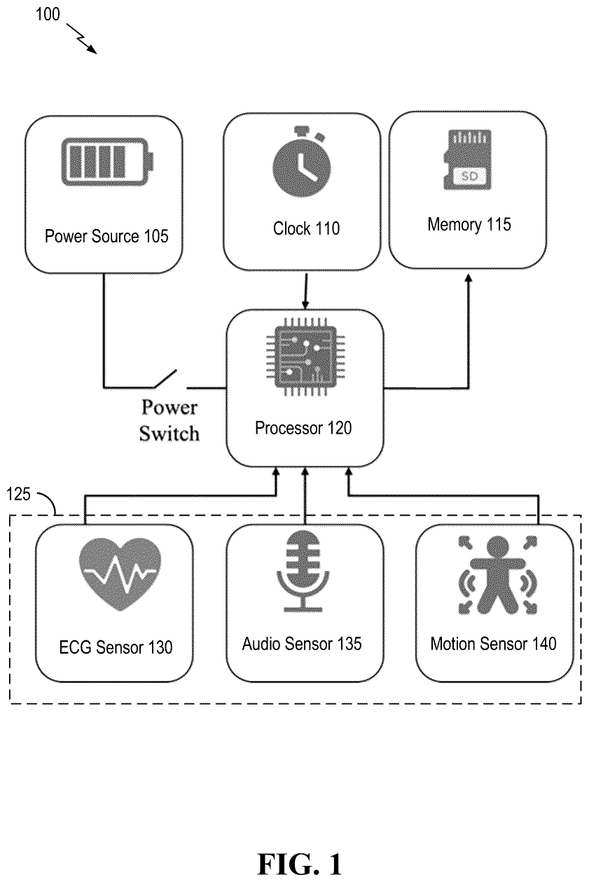

FIG. 1 is a block diagram conceptually illustrating a wearable infant monitoring system 100. As shown in FIG. 1, a processor 120 can receive data representing an acquired signals 125 from an ECG sensor 130, an audio sensor 135, a motion sensor 140, and/or similar sensors. The signals 125 can be sent to the processor 120 directly, or sent from a remote location (e.g., data collected from a remote sensor) via a communication network. In some examples, the wearable infant monitoring system 100 may communicate with Bluetooth Low Energy (BLE) to reduce energy consumption.

In some embodiments, all or some of the components illustrated in FIG. 1 may be arranged in a housing. The housing for the wearable infant monitoring system 100 may be dimensioned to fit within a pocket of a garment, such as a chest pocket of a shirt. In some embodiments, the housing may be dimensioned to fit within a pocket of a child garment, such as a garment for an infant, toddler, or you child. A garment may include any clothing item sized to fit a child, such as a newborn through 12, 18, 24 months of age, a toddle aged between 2-4 years, a child aged between 4-6 years, or the like. For example, in some embodiments, an infant garment may include any clothing item sized to fit an infant having an average weight of about 5 pounds to about 24 pounds, and/or an average length of about 18 inches to about 32 inches. In order to fit within a pocket of a garment, in some embodiments, the housing may have a profile that is slim enough to fit within a pocket. For example, the housing may have a width and/or length of about ⅛ inch, ¼ inch, ½ inch, ¾ inch, etc. The width and length of the housing may be determined based on the desired garment in which the housing is to be placed. In some embodiments, the housing may be placed within a pocket disposed on an infant's garment. For example, the pocket may have a width ranging from 1 inch to 5 inches, and/or a height of 1 inch to 5 inches. The width and height of the pocket may vary by type of shirt, design of shirt, size of shirt, or the like.

In some embodiments, the wearable infant monitoring system 100 may be free from any affixing features. Affixing features can include any component or accessory that allows the system, or part of the system, to be attached to a user. For example, affixing features can include straps, wires, adhesives, fasteners, bands, connectors, buckles, and the like. For example, a garment pocket may retain the housing via a pocket-fastener, such as a zippered pocket, a flap with a hook-and-loop fastener, or the like.

In some embodiments, the processor 120 can include or comprise any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), a cloud resource, etc. As illustrated in FIG. 1, the processor 120 may be connected to a power source 105 and a clock 110. In some embodiments, the processor 120 may be an ARM Cortex MO processor, connected to a clock 110 set at 48 MHz. The processor 120 may support data reading and writing using Serial Peripheral Interface (SPI), Inter-Integrated Circuit Interface (I2C), Inter-IC Sound (I2S) and Analog to Digital Converter (ADC). For example, the processor 120 may collect data from sensors (e.g., ECG sensor 130, audio sensor 135, motion sensor 140, etc.), storing data on the microSD card (e.g., memory 115), and pooling time from the real-time clock using the I2C and SPI. In some examples, the processor 120 may further include an internal clock.

In some examples, the power source 105 may include a battery, such as a rechargeable battery. For example, the power source 105 may be a 500 mAh LiPo rechargeable battery (LP303450) that provides approximately 11 hours of operational capability per charge. This battery comes with a Protection Circuit Module and meets national (UL2054) and international (IEC 62133) safety standards, including RoHS-Compliance. The system is powered on and off by a manual switch for easy usage.

The signals 125 may correspond to one or more sensor modalities, such as a microphone, a 3-lead ECG sensor, and/or an IMU. In some examples, the audio sensor 135 may be a breakout board, such as a SPH0645LM4H breakout board, which includes a single MEMS microphone and circuitry to output digital signals (e.g., 24-bit data) using an I2S protocol. Moreover, for example, the audio sensor may have a low current requirement and high Signal-to-Noise-Ratio (SNR). For example, the current requirement may be 600 μA and the SNR may be 65 dB. In some examples, the ECG sensor 130 may be a heart rate monitor, such as an AD8232 monitor, which measures the electrical activity of the heart and outputs an ECH as an analog reading. In some examples, the ECG sensor 130 may be attached to a user via electrodes. The electrodes may include disposable electrodes, such as electrodes connected via button snaps. Furthermore, in some examples, the motion sensor 140 can include one or more gyroscopes configured to measure one or more axes of movement. For example, the motion sensor 140 may include a 3-axis accelerometer, a 3-axis magnetometer, and a 3-axis gyroscope. In other examples, the motion sensor 140 may be configured as an inertial measurement unit (IMU). These sensors together can provide data on acceleration, direction, and orientation, respectively.

The wearable infant monitoring system 100 can further include, or be connected to, a memory 115. The memory 115 can include or comprise any suitable storage device(s) that can be used to store suitable data (e.g., audio data, motion sensor data, etc.) and instructions that can be used, for example, by the processor 120. In some examples, the memory 115 may include 256 KB of flash memory and 32 KB of RAM. Moreover, in some examples, besides the flash memory in the microcontroller, to store the collected data, the memory 115 may include a 32 GB microSD card. The microSD socket may connect to inputs of a corresponding microcontroller via SPI port pins. In some examples, the memory 115 may utilize an SD card that uses an exFAT format to maximize the read-write speed. For example, a 32 GB SD card may record up to a total of 65 hours of audio, motion, and ECG data across multiple recordings.

The memory 115 may be a memory that is “onboard” the same device that receives the signals 125, or may be a memory of separate device connected to the wearable infant monitoring system 100. Methods for performing health status detection may operate as independent processes/modules or a specialty processor (such as a GPU) that achieves greater efficiency in processing the signals 125, as described below. The memory 115 can include any suitable volatile memory, non-volatile memory, storage, or any suitable combination thereof. For example, memory 115 can include random access memory (RAM), read-only memory (ROM), electronically-erasable programmable read-only memory (EEPROM), one or more flash drives, one or more hard disks, one or more solid state drives, one or more optical drives, etc.

### Example Processes

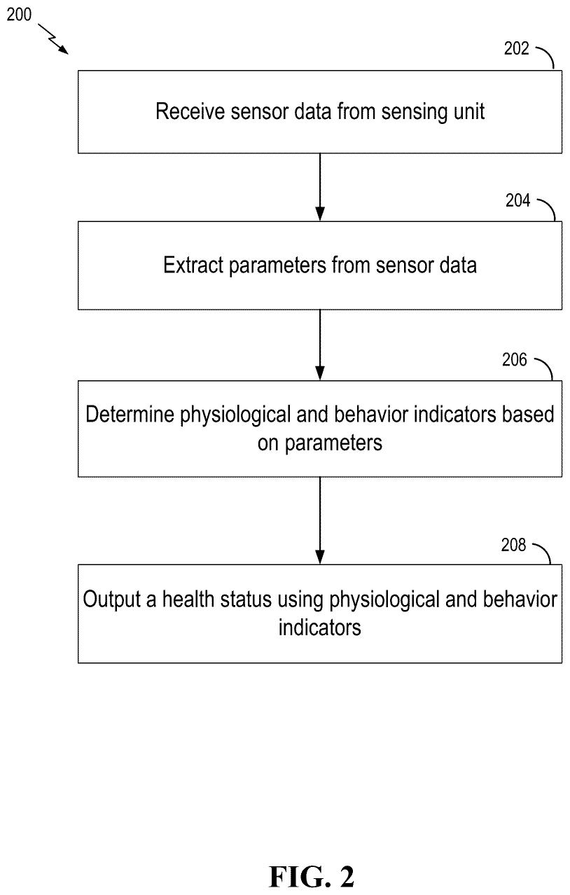

FIG. 2 is a flow diagram illustrating an example process 200 for performing health status detection. In some examples, the health status may correspond to a state of an infant. As described below, a particular implementation can omit some or all illustrated features/steps, may be implemented in some embodiments in a different order, and may not require some illustrated features to implement all embodiments. In some examples, an apparatus (e.g., wearable infant monitoring system 100, etc.) in connection with FIG. 1 can be used to perform all or part of example process 200. However, it should be appreciated that other suitable processing hardware for carrying out the operations or features described below may perform process 200.

At process block 202, sensor data is received from a sensing unit. In some examples, the values within the sensor data may be static or dynamic. The sensor data may be obtained from one or more sensors, such as an ECG, an inertial measurement unit (IMU), an audio sensor, or the like. For example, the sensor data may include measurements such as acceleration, orientations, angular rate, and/or gravitation forces obtain from an IMU.

At process block 204, parameters are extracted from the sensor data. As described below, the analysis may include extracting one or more cardiac parameters from ECG data, detecting environmental sounds, infant sounds, and caregiver interactions associated with the audio sensor, and/or determining an activity associated with the IMU data. For example, the audio sensor may detect noises produced directly by an infant, as well as environmental sounds, such as ambident noises, noises produced by bystanders (i.e., a parent), noises from traffic, noises from household appliance, or the like. In other non-limiting examples, parameters may be extracted using artificial intelligence, a deep learning algorithm, a machine learning model, etc. trained on identifying and analyzing parameters of interest within ECG, IMU, and/or audio data.

At process block 206, physiological and behavior indicators and determined based the extracted parameters. In some examples, the physiological and behavior indicators may be determined by comparing the parameters with one or more clinical threshold values. For example, the one or more threshold values may be associated with an average interbeat interval, R-R peak, heart rate, etc.

At process block 208, a health status is outputted using the physiological and behavior indicators. In some examples, the output may further include a probability of an event or condition based on the health status. As just one example, as will be further described, a wearable sensor system may implement the above-described process. The system may be configured to collect or receive physiological or other patient data, may be able to generate and/or communicate reports. In some instances, the monitor may be able to determine recommendations for treating/addressing the determined health status. Such system may be configured to provide one or more, or even all of these functions.

### Neural Network Implementation

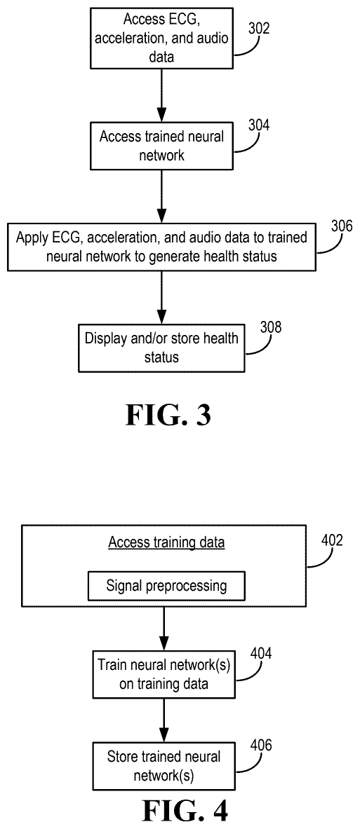

Referring now to FIG. 3, a flowchart is illustrated as setting forth the steps of an example method for performing health status detection using a suitably trained neural network or other machine learning model. As will be described, the neural network or other machine learning model takes ECG data, acceleration data, and audio data (e.g., data received from the wearable infant monitoring system 100) as input data and generates a health status as output data.

### Accessing Data

The method includes accessing ECG data, acceleration data, and audio data with a computer system, as indicated at step 302. Accessing the ECG data, acceleration data, and audio data may include retrieving such data from a memory or other suitable data storage device or medium. Additionally, or alternatively, accessing the ECG data, acceleration data, and audio data may include acquiring such data with an ECG sensor, a motion sensor, and an audio sensor, respectively, and transferring or otherwise communicating the data to the computer system, which may be a part of a wearable medical device that includes the ECG sensor, motion sensor, and/or the audio sensor. As described below, the ECG data may include R-R peaks, interbeat intervals, and heart periods.

### Accessing Trained Neural Network

A trained neural network (or other suitable machine learning model) is then accessed with the computer system, as indicated at step 304. In general, the neural network is trained, or has been trained, on training data in order to evaluate a user's health status. This evaluation is achieved, in part, by the neural network (or other machine learning model) being trained via an ECG dataset, an audio dataset, and/or a motion dataset. For example, the training dataset may comprise ECG signals captured from an ECG sensor, which may be annotated with the ground truth measures.

The trained neural network can include a neural network with any suitable neural network architecture for generating a health status. As one non-limiting example, the trained neural network may include a convolutional neural network, such as a convolutional neural network comprising feature extraction subnetworks for passive and active sound signal analysis, and feature generation subnetworks for active sound signal analysis. The trained neural network may in some instances have multiple inputs (e.g., corresponding to sound signals and other sensor data).

Accessing the trained neural network may include accessing network parameters (e.g., weights, biases, or both) that have been optimized or otherwise estimated by training the neural network on training data. In some instances, retrieving the neural network can also include retrieving, constructing, or otherwise accessing the particular neural network architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed. In some examples, accessing the trained neural network may comprise operating a neural network instantiated on a mobile device.

### Applying Data to the Neural Network

The ECG data, acceleration data, and audio data are then input to the trained neural network, generating output as a health status, as indicated at step 306. For example, the health status may comprise a classification of an infant wearing one or more corresponding sensors (e.g., indicating the infant is active, quiet, fussy, drowsy, sleeping, etc.) or a regression of conditions/characteristics associated with the data.

Display and/or Store Output

The health status generated by inputting the ECG data, acceleration data, and audio data to the trained neural network(s) can then be provided to a user, stored for later use or further processing, or both, as indicated at step 308. For example, the health status may be stored on a wearable device memory, may be transmitted to an attached device, uploaded to a server, such as a clinical computer system, displayed on the wearable device (e.g., via an alert to indicate a health status output), etc.

### Neural Network Training

Referring now to FIG. 4, a flowchart is illustrated as setting forth the steps of an example method for training one or more neural networks (or other suitable machine learning models) on training data, such that the one or more neural networks are trained to receive ECG data, acceleration data, and audio data as input data in order to generate a health status as output data. For example, in some implementations, neural network training may be performed on a computer system prior to being instantiated on a wearable device.

In general, the neural network(s) can implement any number of different neural network architectures. For instance, the neural network(s) could implement a convolutional neural network, a residual neural network, or the like. Alternatively, the neural network(s) could be replaced with other suitable machine learning or artificial intelligence algorithms, such as those based on supervised learning, unsupervised learning, deep learning, ensemble learning, dimensionality reduction, and so on.

### Accessing Training Data

The method includes accessing training data with a computer system, as indicated at step 402. In general, the training data can include ECG data, acceleration data, and/or audio data with ground truth annotations generated from ECG data, acceleration data, and/or audio data. Additionally, or alternatively, the accessed training data can include ECG data, acceleration data, and/or audio data received from an example database. Accessing the training data may include retrieving such data from a memory or other suitable data storage device or medium. Alternatively, accessing the training data may include acquiring such data with an ECG sensor, an audio sensor, and/or a motion sensor and transferring or otherwise communicating the data to the computer system.

The method can include assembling training data from ECG data, acceleration data, and audio data using a computer system. This step may include assembling the ECG data, acceleration data, and audio data into an appropriate data structure on which the neural network or other machine learning model can be trained. For example, the machine learning model may include a random forest classifier that operations on ECH data, acceleration data, and/or audio data to identify an infant state. Assembling the training data may include annotating the data. For instance, assembling the training data may include recording or obtaining ECG signals, pre-processing the signals by filtering missing interbeat interval values, or like.

### Training the Neural Network

One or more neural networks (or other suitable machine learning models) are trained on the training data, as indicated at step 404. In general, the neural network can be trained by optimizing network parameters (e.g., weights, biases, or both) based on minimizing a loss function. As one non-limiting example, the loss function may be a mean squared error loss function.

Training a neural network may include initializing the neural network, such as by computing, estimating, or otherwise selecting initial network parameters (e.g., weights, biases, or both). During training, an artificial neural network receives the inputs for a training example and generates an output using the bias for each node, and the connections between each node and the corresponding weights. For instance, training data can be input to the initialized neural network, generating an output as a health status. The artificial neural network then compares the generated output with a ground truth value of the training example in order to evaluate the quality of the a health status. For instance, the sensor data can be passed to a loss function to compute an error. The current neural network can then be updated based on the calculated error (e.g., using backpropagation methods based on the calculated error). For instance, the current neural network can be updated by updating the network parameters (e.g., weights, biases, or both) in order to minimize the loss according to the loss function. The training continues until a training condition is met. The training condition may correspond to, for example, a predetermined number of training examples being used, a minimum accuracy threshold being reached during training and validation, a predetermined number of validation iterations being completed, and the like. When the training condition has been met (e.g., by determining whether an error threshold or other stopping criterion has been satisfied), the current neural network and its associated network parameters represent the trained neural network. Different types of training processes can be used to adjust the bias values and the weights of the node connections based on the training examples. The training processes may include, for example, gradient descent, Newton's method, conjugate gradient, quasi-Newton, Levenberg-Marquardt, among others.

The artificial neural network can be constructed or otherwise trained based on training data using one or more different learning techniques, such as supervised learning, unsupervised learning, reinforcement learning, ensemble learning, active learning, transfer learning, or other suitable learning techniques for neural networks. As an example, supervised learning involves presenting a computer system with example inputs and their actual outputs (e.g., categorizations). In these instances, the artificial neural network is configured to learn a general rule or model that maps the inputs to the outputs based on the provided example input-output pairs.

### Storing Neural Network(s) for Later Use

The one or more trained neural networks are then stored for later use, as indicated at step 406. Storing the neural network(s) may include storing network parameters (e.g., weights, biases, or both), which have been computed or otherwise estimated by training the neural network(s) on the training data. For example, storing the neural network may include instantiating the neural network on a wearable device, such by programming a neuromorphic computer or storing neural network parameters in a wearable device storage system, controller, etc. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

### Computer Systems

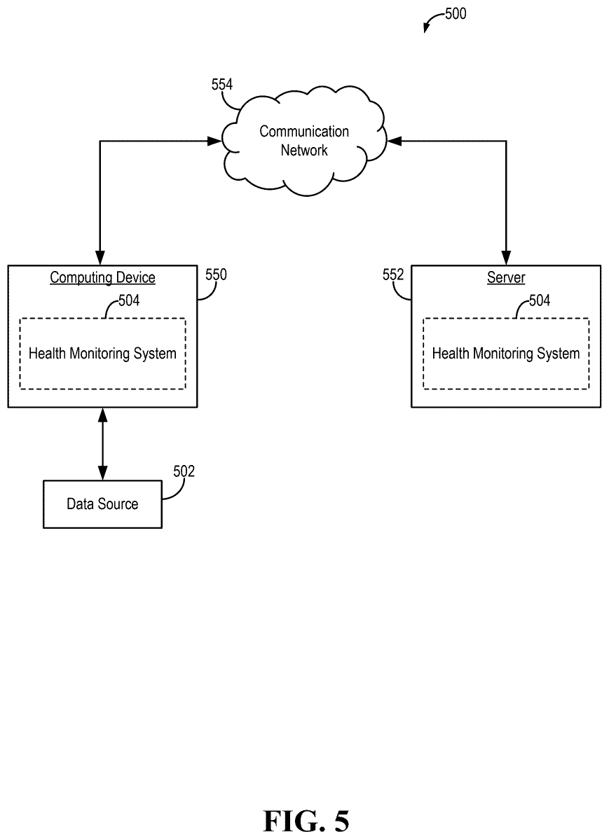

FIG. 5 shows an example of a system 500 for performing health status detection using a health monitoring system in accordance with some embodiments described in the present disclosure. As shown in FIG. 5, a computing device 550 can receive one or more types of data (e.g., ECG data, audio data, motion data, including raw signals) from data source 402. For example, computing device 450 may comprise a wearable device (including one or more wearable sensors) and data source 402 may comprise a microphone, an accelerometer, an electrode patch, or other source of the signal data. In some embodiments, computing device 450 can execute at least a portion of wearable monitoring system 404 to a health status (e.g., a detected status of an infant) from data received from the data source 402.

Additionally, or alternatively, in some embodiments, the computing device 550 can communicate information about data received from the data source 502 to a server 552 over a communication network 554, which can execute at least a portion of the health monitoring system 504. For example, server 552 may comprise a mobile device connected to a wearable device, a mobile application, or a remote server in a clinic. In such embodiments, the server 552 can return information to the computing device 550 (and/or any other suitable computing device, such as a mobile device) indicative of an output of the health monitoring system 504.

In some embodiments, computing device 550 and/or server 552 can be any suitable computing device or combination of devices, such as a wearable computer, a smartphone, a desktop computer, a laptop computer, a tablet computer, a server computer, a virtual machine being executed by a physical computing device, and so on.

In some embodiments, data source 502 can be any suitable source of data (e.g., recorded sound data, cardiac readings, accelerometer data, etc.) another computing device (e.g., a server storing ECG data, audio data, motion data, spectrogram data, etc.), and so on. In some embodiments, data source 502 can be local to computing device 550. For example, data source 502 can be incorporated with computing device 550 (e.g., computing device 550 can be configured as part of a device for measuring, recording, estimating, acquiring, or otherwise collecting or storing data). As another example, data source 502 can be connected to computing device 550 by a cable, a direct wireless link, and so on. Additionally, or alternatively, in some embodiments, data source 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 554 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, and so on.

### Examples and Experiments

Described below are experimental setups and validations of the disclosed system and methodology. In some examples, the approach described herein may be used to a health status of a user wearing a sensor system, such as the wearable infant monitoring system 100.

Wearable sensor systems involve multiple steps, including (a) data acquisition, i.e., the collection of the raw sensor data, (b) data processing, i.e., reduction of the raw sensor data into desired features and metrics, (c) health status detection, i.e., comparing reduced data or metrics against clinical thresholds for diagnostic or treatment purposes, (d) wireless communication, i.e., transfer of data metrics and clinical information to physicians, parents and/or other health professionals, and (e) power supply, which is an essential consideration underlying the successful implementation of all other parts of the system. In some examples, there may be a need for digital health technologies, including wearables, to incorporate technical validation, analytic validation, clinical validation, and usability.

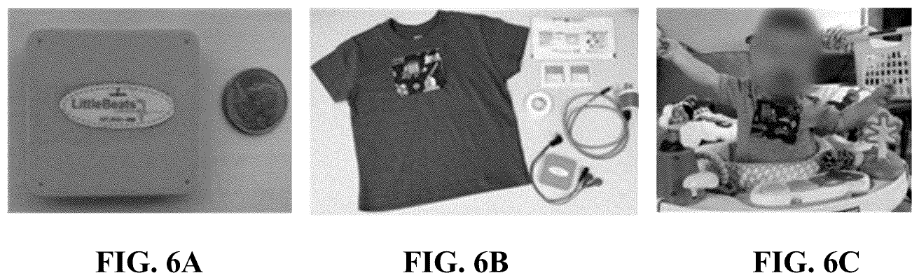

To collect synchronized multi-modal sensor data suitable for infants and young children, a unique sensing platform was designed. All electronics are housed in a 3D-printed case (55×57×13 mm; see FIG. 6A), and the device weighs 1.48 ounces (42 grams), making it suitable as a child-wearable. The device is placed in a specially designed t-shirt that the child wears (see FIG. 6B). A chest pocket with side opening is centered on the shirt and snugly holds the device in place. The inside of the pocket is padded with a thin foam layer for comfort, and two ¼ inch metal snaps are used to securely close the pocket.

Custom firmware was developed for the system written in C programming language and enable timestamped data streams from all three sensor modalities to be stored on the SD card. For both adult and infant data (including daylong home recordings not reported here), audio was sampled at 22 kHz (down sampled to 16 kHz during pre-processing), ECG at 2426 Hz, and 9-axis motion data at 70 Hz. Writing of audio data to the SD card occurs every 10 seconds, whereas writing the ECG and IMU data occurs every 30 seconds. These “chunk” durations were determined, keeping the maximum data transfer rate of the peripheral bus of the processor (which is a 32-bit multi-central/multi-peripheral bus) in mind.

The time from the RTC is recorded at the start and end of each data chunk for synchronizing the multiple data streams. These data are stored in Little-Endian binary format unreadable to humans without further processing. These binary files are converted to human-readable format (.csv for ECG and IMU; .wav for audio) with custom Python scripts after removing the SD card from the device. The data extraction codes also perform several pre-processing steps (described in bellowed) to verify and maintain the quality of the data.

Using the time-keeping unit, the data collected was synchronized from three modalities. As these files are written to the SD card in an asynchronized manner, and the sampling rate of each modality is different, there is a need for synchronization. As described herein, each file (or data chunk) in the SD card is timestamped with start and end times. The recorded samples was split into frames (files) by aligning the starting index to the timestamp. The split sample frames are naturally synchronized because the UTC timestamps are consistent across the three sensor modalities. Depending on the version of the device firmware used during data collection, the split sample frames was zero-padded for ECG and audio data prior to synchronization to match the predicted frame period. IMU data, which are collected at a much lower sampling rate, were not affected by missing samples.

Importantly, data collection for the various studies described in this report was slowed due to the COVID-19 pandemic, and in the interim, the device firmware was updated. Studies 1, 3, and 4 reported below used Version 1 firmware, whereas Studies 2 and 5 used Version 2. The key update to Version 2 was switching from FAT32 (write speed: 108.42 KB/sec) formatting of the SDcard to exFAT (497.33 KB/sec) format and the corresponding SdFat Arduino library, which resulted in faster write times and, thus, substantially fewer missing samples in audio (see Study 4 versus 5) and no missing samples in ECG (see Study 1 versus 2).

Sixteen adult participants (56.3% female; Mean age=27.4 years, SD=8.82, Range: 18-46) were recruited through a university listserv and flyers displaying study information posted in multiple university buildings. Both forms of recruitment reach adults across various educational and racial/ethnic backgrounds. Participants reported on their highest level of education (13% high school graduate, 20% some college, 40% bachelor's degree, 27% advanced degree) and their race and ethnicity (33.3% Asian, 60% White non-Hispanic, 6.7% Hispanic). Participants were eligible to participate if they met the following criteria: (a) at least 18 years of age, and (b) no known heart problems or abnormalities.

Participants visited the laboratory and were guided through a series of tasks while wearing two ECG monitors: (a) Device (Version 1 firmware) and (b) the BIOPAC MP160 system (BIOPAC Systems, Santa Barbara, CA). Six disposable, pre-gelled, signal-conditioning electrodes were placed on the participant (3 electrodes per device): two below the left clavicle, two below the right clavicle, and two just below the ribcage (i.e., Einthoven's triangle). Pairs of electrodes were placed side by side but did not touch or overlap. The device and BIOPAC BioNomadix wireless transmitter were placed in a specially designed t-shirt with two chest pockets, providing a form factor that was comparable across the two devices and mirrors the form factor used with infant and child participants. BIOPAC samples ECG at 1,000 Hz.

Participants were video recorded while completing the following tasks: (a) 3-minute baseline, which involved viewing a clip from a calming video of sea animals, (b) 4-min puzzle task, which involved solving a 14-piece Tangram puzzle, (b) 2-min recovery using another clip from the video viewed during the baseline session, and (d) 4-min nonverbal abstract reasoning task using Ravens' Progressive Matrices (Standard version). The puzzle and matrices tasks each presented a cognitive challenge, and such tasks have been used successfully in prior research to elicit a physiological stress response (i.e., cardiac vagal withdrawal) among adults and children alike. Further, participants completed the two challenge tasks (i.e., puzzle and matrices) while a large countdown timer was displayed on the computer screen, thereby increasing potential stress. For the Tangram puzzle task, eight participants completed the puzzle in under 4 minutes (M=2.63, SD=0.87), and ECG data for these participants included only the time in which the participant was engaged in solving the puzzle. The Raven's Progressive Matrices include sixty multiple choice items; items are organized within five sets (twelve items each), and items within each set increase in difficulty. Participants were instructed to complete as many items as possible within the time allotted and no participants completed all items within the 4-min timeframe (M items completed=28.31, SD=6.22).

The following data pre/post-processing steps were implemented to extract IBI values from the ECG Device and BIOPAC data and compute RSA values: (1) CardioPeak & Segmenter was used to extract the R-R peaks from the Device and BIOPAC ECG data, and derive the time in milliseconds between consecutive R peaks (i.e., IBI values, 250 Hz sampling rate); this software outputs separate IBI files for each task/session (task time information, which was derived for the BIOPAC and The device from the video and audio recordings respectively, provided in a separate CSV file serves as an additional input file); (2) to correct for artifacts due to zero-padding (M=2.36% missing samples, SD=0.14%) in Version 1 of the device firmware, the IBI data was passed through a custom filtering script that took into account missing data samples and used standard IBI artifact detection and editing approaches to correct IBI points due to missing samples; (3) The device and BIOPAC IBI for each task were manually aligned in time by plotting IBI values from each device as a function of time in Excel; (4) all IBI data files were reviewed and, when needed, manually edited using CardioEdit v1.5, by the inventors who had trained and certified by the Porges' Brain-Body Center for Psychophysiology and Bioengineering (BBCPB) at the University of North at Carolina Chapel Hill; (5) RSA was computed from BIOPAC and the device IBI data using the Porges-Bohrer algorithm by calculating the natural logarithm of the variance of heart period within the frequency bandpass related to respiration (0.12-0.40 Hz for adults) in CardioBatch Plus software. Within each task, RSA values were computed in 30-sec epochs and then averaged across epochs to obtain task-level means.

Data from an additional 7 participants were collected but were excluded because for one or more of the target sessions (baseline, puzzle, recovery, matrices), the BIOPAC file could not be edited due to an extreme value and/or more than 5% edits (n=4), technical problems with the video recording, which may be needed to align the two files at the session level (n=2), and fewer than 90 seconds of data available (n=1).

The error statistics was computed in the device IBI values via (a) mean error (i.e., average difference between BIOPAC and the device IBI values), (b) mean absolute error (i.e., average absolute difference between BIOPAC and the device IBI values), and (c) mean absolute percent error (i.e., MAPE; mean of absolute error divided by BIOPAC IBI value and multiplied by 100). MAPE is a widely used metric in validation of physiological sensors, and an error rate of ±10% has been deemed acceptable for ECG-related measurements in recent studies and by the Consumer Technology Association. The number of total IBI data points and error statistics for each task are shown in Table 1.

TABLE 1
Error statistics and Bland-Altman analyses for adult participants' interbeat
intervals (milliseconds) during the baseline, puzzle, recovery, and matrices tasks.

| | Absolute | | Mean error | Bland-Altman analysis | |
|---|---|---|---|---|---|
| Session (n observations) | Mean Error | MAPE (%) | (SD) | Lower LoA | Upper LoA |
| Baseline (n = 3355) | 49.6 | 5.93% | 11.1 (77.3) | −162.54 | 140.33 |
| Tangram puzzle (n = 3744) | 41.9 | 5.29% | 4.5 (62.8) | −127.59 | 118.65 |
| Recovery (n = 2777) | 49.7 | 5.97% | 12.7 (73.1) | −156.02 | 130.55 |
| Matrices (n = 4589) | 45.6 | 5.62% | 10.6 (68.3) | −144.51 | 123.28 |

The MAPE was under 6% for all tasks across all participants. MAPE values were also computed separately by participant and ranged from 0.57% to 13.64% for baseline, 0.59% to 11.74% for the puzzle task, 0.57% to 11.31% for recovery, and 0.63% to 12.39% for the matrices task. Of the 64 MAPE scores (16 participants×4 tasks), 26 were under 5%, 33 were under 10%, and 5 were between 10% and 13.64% percent. Data from the same participant yielded the lowest MAPE values across all tasks, whereas data from two participants yielded the highest MAPE values (baseline and matrices for one participant; puzzle and recovery for the other). For descriptive purposes, the bivariate correlational between BIOPAC average IBI values and MAPE scores were computed. Weak-to-moderate positive associations emerged, although associations were not statistically significant (rs=0.24, 0.45, 0.26, 0.21, ps=0.37, 0.08, 0.33, 0.44, baseline, puzzle, recovery, and matrices tasks respectively). Scatterplots of these associations indicated a positive association between BIOPAC IBI average scores and MAPE until IBI scores reached approximately 0.90 seconds; the few cases with an average IBI score greater than 0.90 seconds showed no discernible increase in MAPE.

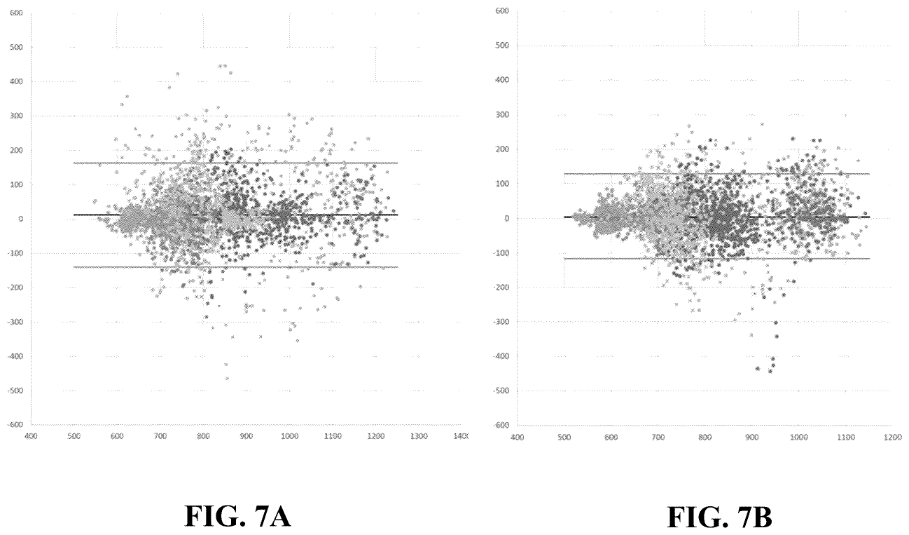

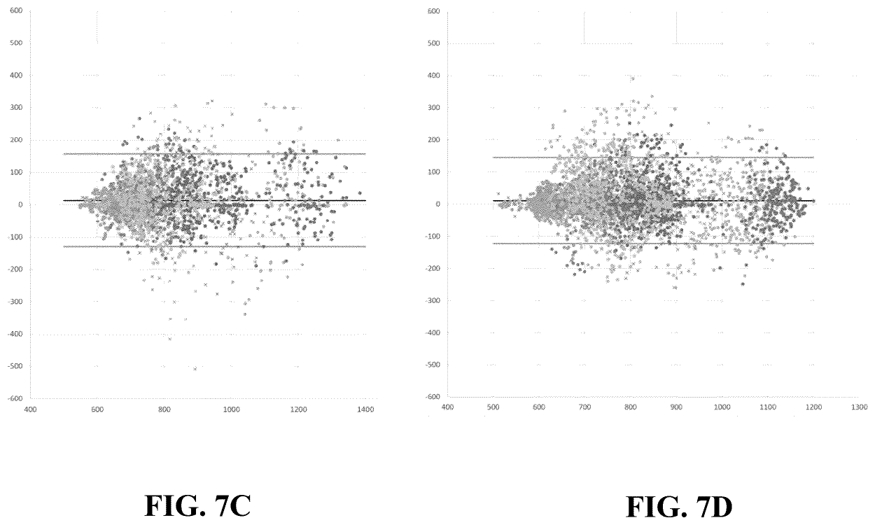

Second, Bland-Altman plots provide a direct and appropriate comparison between quantitative measurements of the same phenomenon. Bland-Altman plots of IBI values, in which the X axis represents the mean of the two measurement instruments (the device, BIOPAC) and the Y axis represents the difference (in milliseconds) between the two instruments (BIOPAC minus the device), are shown in FIGS. 7A-C. IBI values are plotted separately by task and color coded by participant. Bland-Altman plots can be used to assess the presence of outliers (with respect to differences in the two measurements) or whether data are systematically biased (i.e., difference between measures is consistently in one direction). Across tasks, the mean error (BIOPAC-device) in IBI values ranged from 4.5 milliseconds (puzzle task) to 12.7 milliseconds (recovery) as shown in Table 1 above, indicating the BIOPAC and the device IBI values were typically within hundredths or thousandths of a second and that, on average, the device (vs. BIOPAC) IBI values were slightly lower. The Bland-Altman plots also show that 95% of the BIOPAC-device errors (difference scores) fall within a range of approximately ± of 150 milliseconds (see Table 1 for specific 95% limits of agreement for each task). Further, errors are smaller at the lower end of observed IBI values (i.e., ˜500 to ˜700 milliseconds on the X axis) and are more dispersed at the middle and higher ends of observed IBIs (i.e., ˜800 to ˜1200 milliseconds), although this pattern varies as a function of case and task (e.g., the case shown in peach exhibits moderate levels of IBI, with lower error rate in baseline task but more dispersion in errors in puzzle, recovery and matrices tasks). Finally, errors show a relatively even distribution around the mean error (black line) and limits of agreement (orange lines) in the Bland-Altman plots across tasks and individuals, indicating little systematic bias in the errors.

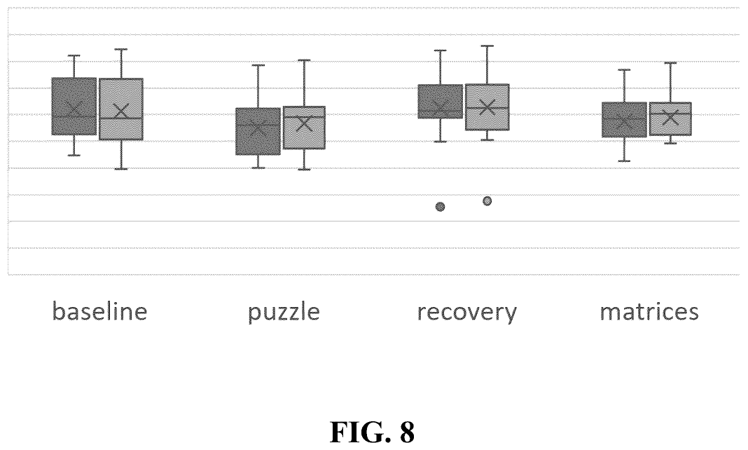

The third analysis focused on RSA measurements derived from the IBI data (as described above). The RSA sample means and distributions were plotted for each task (see FIG. 8). Because the puzzle and matrices tasks each presented a mild to moderate challenge, RSA may decrease from baseline to the puzzle task, increase from puzzle to recovery, and decrease again from recovery to the matrices task. Paired t-tests indicated significant (p<0.05, one-tailed) and hypothesized differences in RSA means across tasks: (a) baseline minus puzzle, t(15)=2.71 and 1.78, p=0.008 and 0.047, BIOPAC and the device respectively, (b) puzzle minus recovery, t(15)=−2.30 and −1.96, p=0.018 and 0.034, and (c) recovery minus matrices, t(15)=2.36 and 2.00, p=0.016 and 0.031. Thus, despite a degree of error in the device IBI values, task-related changes in RSA were observed and mirrored RSA changes assessed via IBI data obtained from the BIOPAC system.

Five infants were recruited (3 females, M_{age}=7.64 months, age range: 4-12 months) via an announcement posted on a university-wide listserv. Paralleling Study 1 procedures, infant ECG data were collected simultaneously by the device and BIOPAC MP 160 system in the laboratory. Due to the burden of wearing two ECG monitors simultaneously and because results from Study 1 results indicated acceptable agreement between the two devices, the infant sample was limited to five participants across a wide range of ages during the first year of life. Infants were eligible to participate if they met the following criteria: (a) under 12 months of age, (b) no known cardiac abnormalities, and (c) their mother was willing to speak English during the visit if English was not her native language. All ECG data collected are included in the analyses below.

Infant-mother dyads participated in a laboratory visit, in which infants wore the device (Version 2 firmware) and BIOPAC ECG sensors. The device and BioNomadix wireless transmitter were placed in dual chest pockets of a specially designed infant shirt. While seated on their mother's lap, infants were videorecorded during a 3-min baseline session that was identical to the baseline video session used in Study 1. Following the baseline session, infants and mothers were observed in the Still Face Paradigm (SFP), which consisted of three 2-minute episodes: (1) play, while infant was seated in bouncy seat or high chair (depending on age), (2) still face, in which mothers were cued (via a brief knock on the playroom door) to cease verbal and physical interaction with their infant while looking at the infant with a neutral face, and (3) reunion, in which mothers were cued (via brief knock) to resume interacting with their infant. No toys were present during the SFP, and mothers were asked to not take their infant out of the seat. The still face episode of the SFP is emotionally challenging for infants and typically elicits a distress response. If the infant displayed high levels of prolonged distress (i.e., 15-20 seconds) during the still face episode, the episode was curtailed. Mother-infant interaction during the SFP was videorecorded via two remote-controlled cameras with pan/tilt/zoom features; the cameras were mounted on opposite corners of the playroom and controlled from an adjacent observational booth.

Processing of the BIOPAC and the device ECG, IBI, and RSA data were identical to the steps outlined in Study 1 with the following exceptions. First, Version 2 of the device firmware results in no missing ECG samples and, thus, the custom filtering script that automated correction of IBI points was not implement due to missing samples described in Study 1 (Data Processing; Step 2). Second, in computing RSA values for the infant data, the natural logarithm of the variance of heart period within the frequency bandpass related to respiration for infants (i.e., 0.3-1.3 Hz) was calculated in CardioBatch Plus software.

The same error statistics reported in Study 1 (i.e., mean error, mean absolute error, MAPE). As shown in Table 2, the MAPE was under 2% for all tasks across all participants. Within participant, MAPE ranged from 0.86% to 1.54% for baseline, 0.74% to 1.10% for the SFP play episode, 0.82% to 3.65% for SFP still episode, and 0.69% to 2.23% for SFP reunion episode. Of the 20 MAPE scores (5 participants×4 tasks), 9 were under 1%, 9 were under 2%, and 2 scores were 2.23% and 3.65%, respectively.

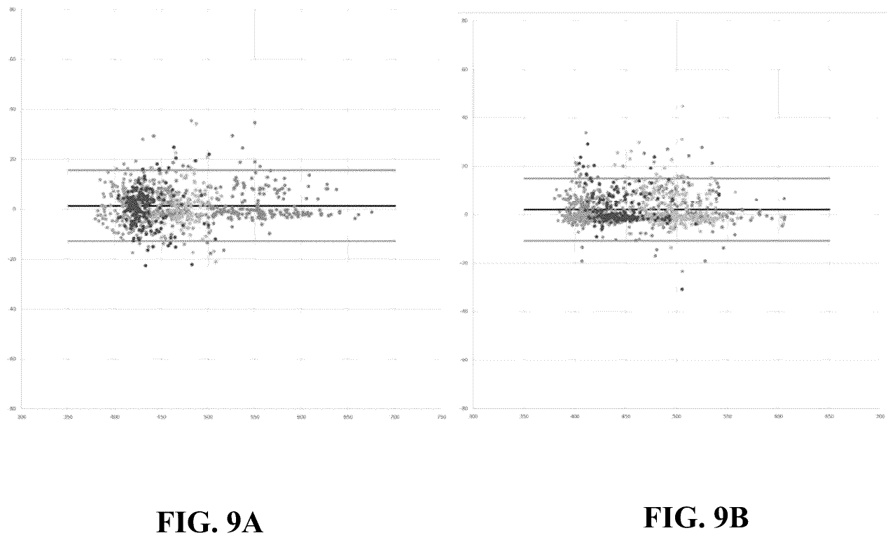

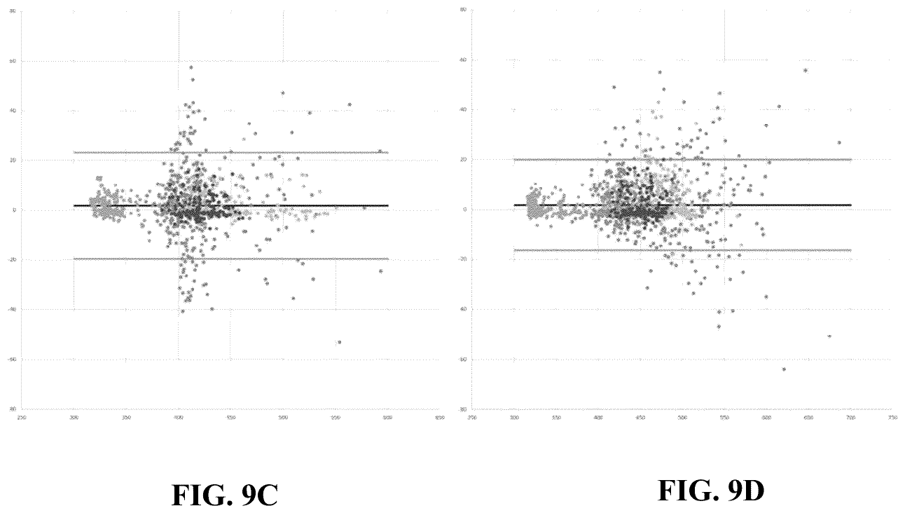

Next, Bland-Altman plots (by task and color coded by participants) are shown in FIGS. 9A-9D, with corresponding statistics reported in Table 2. The mean error (BIOPAC-device) in IBI values ranged from 1.3 milliseconds (baseline) to 2.0 milliseconds (SFP play), indicating the BIOPAC and the device IBI values were typically within thousandths of a second and that, on average, the device values were slightly lower than BIOPAC values. The Bland-Altman plots also show that 95% of the BIOPAC-device errors (difference scores) fall within an approximate range of ±15 to 20 milliseconds (see Table 2 and FIGS. 9A-9D for specific 95% limits of agreement for each task). Further, for the baseline and SFP play episode, errors are consistently small and approach zero cross the range of observed IBI values (˜375-675 milliseconds on the X axis). For the SFP still and reunion episodes, the errors, although still relatively small, are more dispersed, particularly at the higher end of the observed IBI values (i.e., ˜450 to ˜650 milliseconds). Finally, across tasks and individuals, the errors are distributed relatively evenly around the mean error (black line) and limits of agreement (orange lines), suggesting little systematic bias in the ECG/IBI data derived from the device.

TABLE 2
Error statistics and Bland-Altman analyses for infant participants' interbeat
intervals (milliseconds) during the baseline and SFP play, still, and reunion episodes.

| | Absolute | | Mean error | Bland-Altman analysis | |
|---|---|---|---|---|---|
| Session (n observations) | Mean Error | MAPE (%) | (SD) | Lower LoA | Upper LoA |
| Baseline (n = 907) | 5.4 | 1.17% | 1.3 (7.22) | −15.45 | 12.84 |
| SFP play episode (n = 1075) | 4.4 | 0.96% | 2.0 (6.58) | −14.87 | 10.93 |
| SFP still episode (n = 936) | 6.9 | 1.66% | 1.7 (10.93) | −23.09 | 19.75 |
| SFP reunion episode (n = | 5.6 | 1.22% | 1.7 (9.29) | −19.92 | 16.49 |
| 1472) | | | | | |

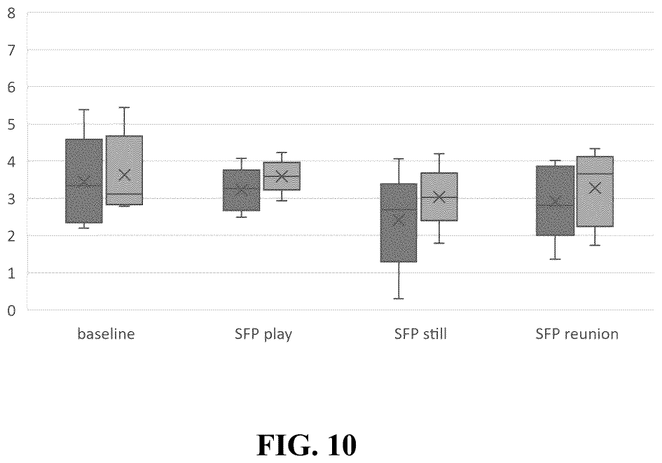

The RSA task mean was examined to assess the pattern of change across baseline and SFP episodes, although the small sample size prohibited statistical tests. Based on a host of prior studies, the highest RSA values may occur during the baseline and SFP play sessions (indicative of low-stress contexts) and lowest RSA values (indicative of RSA withdrawal in response to stressor) may occur during the SFP still episode, with modest increases in RSA during the reunion episode, indicating partial recovery from the stress of the SFP still episode. The RSA sample means and distributions were plotted for each task (see FIG. 10). Although RSA values based on the device data are consistently higher than values from the BIOPAC data, the more important finding is that the device data followed the same task-related changes in RSA observed in the BIOPAC data, indicating sensitivity to within-person changes in RSA.

Twelve adults (66.7% female; Mean age=24.7 years, SD=5.42, Range: 18-33) were recruited through online announcements at a university in a mid-sized midwestern city. Participants reported on their highest level of education (16.7% high school graduate, 25% some college, 41.7% bachelor's degree, 16.7% advanced degree) and their race and ethnicity (66.7% Asian, 33.3% White, non-Hispanic).

Participants wore the device (Version 1 firmware) and the smartphone in two chest pockets of a custom t-shirts, and the smartphone and device each fit snugly in their respective shirt pocket, permitting a comparable form factor. (Note that other more expensive and precise IMUs that are worn with form-fitting chest straps do not permit a parallel form factor.) Participants were video recorded while performing a series of six physical activities (i.e., sit, stand, walk, glide or walk sideways, squat or deep knee bends, and rotating in chair) commonly used in the activity recognition literature. Here, sitting and standing capture the stability of the data, while walking, gliding, and squatting capture acceleration along the 3-different axes of the accelerometer. Rotation captures the performance of the gyroscope. Following are the six task descriptions: (1) The participant sits on a chair and watches a video for 2 minutes; (2) Between each activity, the participant stands for 30 seconds; (3) The participant walks to the end of the room and back three times; (4) The participant glides or steps to the left until they reach the end of the room, then glides or steps to the right until they reach to the other end of the room, for one minute; (5) The participant completes squats or deep knee bends for one minute; and (6) The participant sits in an office chair and rotates slowly five times.

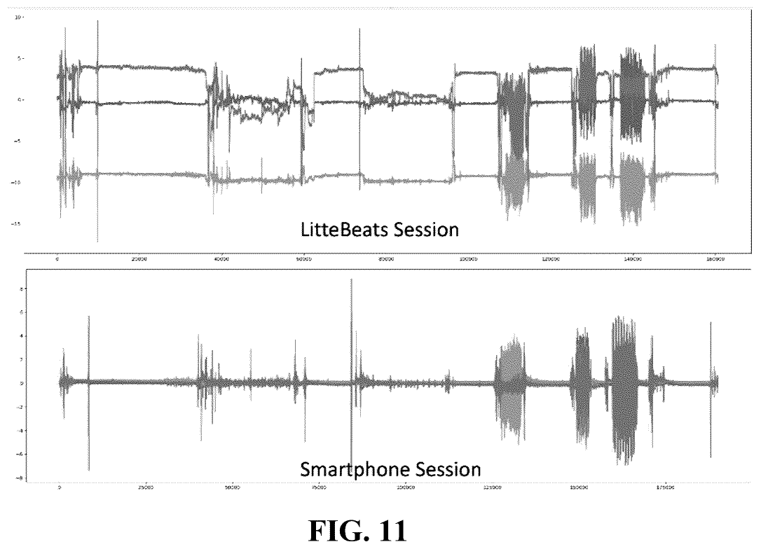

The smartphone uses an IMU data collection app named “SensorLogic” that collects the data and provides processed accelerometer data mitigating the effect of gravity and noise on the IMU as shown in FIG. 11. The microcontroller on the device reads the IMU data directly from the appropriate registers with a function call. The gravitational effect was removed from the device data by subtracting the gravitational acceleration (9.8 m/s) from the affected axis of the accelerometer data. Note that the smartphone performs this action internally.

Due to the asynchronous collection of the IMU data with the ADC, the sampling rate of the IMU data collection is dynamic with an offset of +5 Hz. Because of algorithms used to validate the IMU sensor data and because such traditional machine learning and signal processing algorithms take input with a fixed sampling rate, the dynamic sampling rate of the device may be adjusted. To this end, the timestamp from the time-keeping unit was utilized and a sliding window was used to determine the number of samples in each non-overlapping 30-second interval. Up sampling (with interpolation) or down sampling the 30-seconds was used based on whether there were more or fewer data points than the required sampling rate.

Among the six tasks, five were relevant to assessing the performance of the accelerometer: sit, stand, walk, glide, and squat. Because the accelerometer on the chest fails to differentiate between sitting and standing still, these two activities were combined under a single label (“upright”). 5 s segments were used and each segment was labeled with the activity label, which yields a total 1254 samples across all four activities with the following distribution: 812 upright, 150 walk, 176 glide, and 116 squat. Note that when there is an imbalanced dataset where all classes do not have the same number of samples, and samples are omitted where the participant transitions from one activity to another.

The data was randomly split into train and test sets with 80% training and 20% testing samples, while ensuring that samples from all classes are present in both training and testing datasets. Using 10-fold cross validation, any bias of the train-test split was eliminated. The data was normalized by removing the mean and scaling to unit variance. These normalized samples were used as the input to the classifier.

Each 5 s segment was classified using a multiclass Random Forest classifier for the following four-way classification problem: upright vs. walk vs. glide vs. squat. Random Forest is a meta estimation technique that fits a number of decision trees on multiple sub-samples of the dataset and then takes the average. This averaging increases the prediction accuracy and controls for overfitting. 100 decision trees were used in this random forest and use entropy to measure the quality of a split.

The mean and standard deviation of three metrics were reported across ten data splits to evaluate classification performance. These metrics are: (1) accuracy, which captures the overall level of agreement between the classifier and the ground truth, (2) F1-score, which represents the harmonic mean of precision and recall, where precision (or “positive predictive value”) is the number of true positive predictions divided by the number of all positive predictions and recall (or “sensitivity”) is the number of true positive predictions divided by the number of all true positives, and (3) Cohen's kappa. Chance (a classifier that assigns labels uniformly at random) would achieve an accuracy of 25%, an F-1 score slightly below 25% (because of class imbalance), and a kappa value of 0.0. Kappa values between 0.60 to 0.80 indicate moderate agreement and are considered acceptable; kappa values greater than 0.80 indicate substantial agreement and are considered excellent.

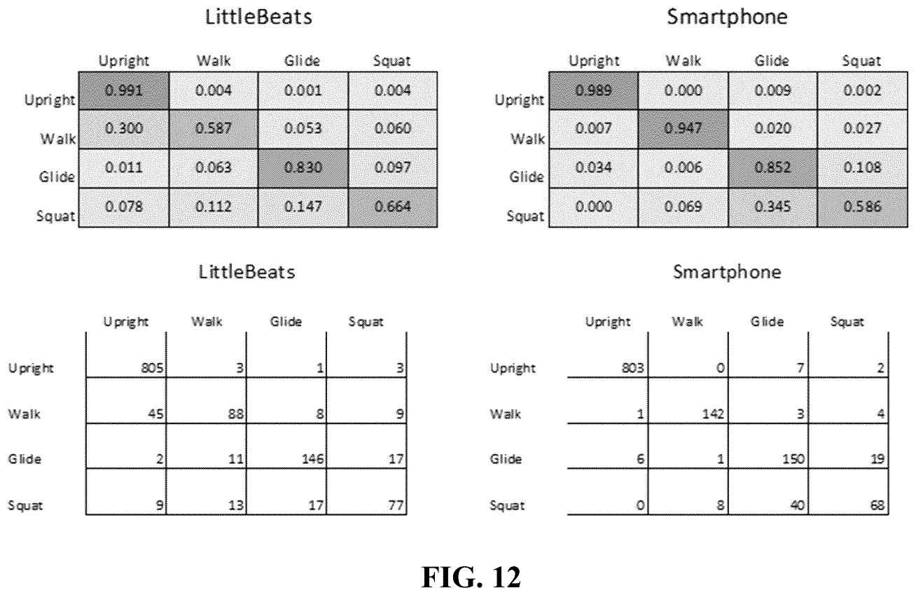

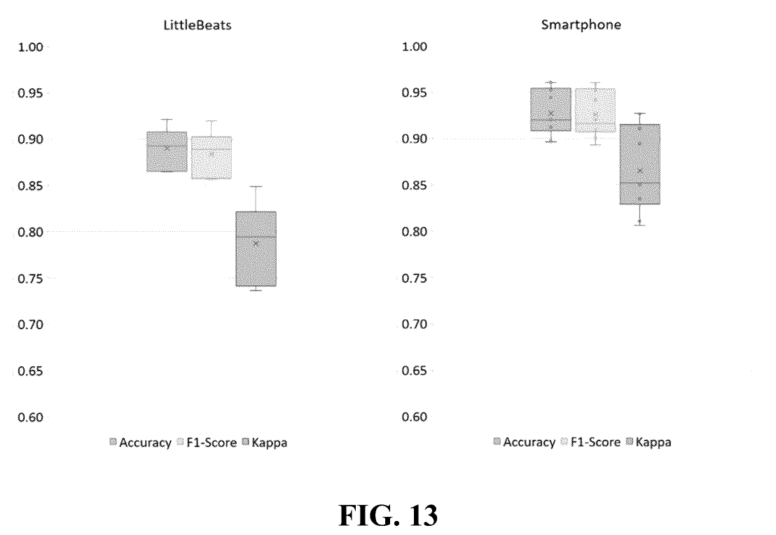

FIG. 12 shows the confusion matrices for the four-way activity classification separately for the device and smartphone data, and FIG. 13 shows the related performance metrics. Although the algorithm for activity detection performed better when applied to data from the smartphone, the performance metrics on the device data also showed high levels of accuracy (89%), F1-score (88%), and Cohen's kappa (0.79) and are therefore on the boundary between acceptable and excellent. Further, the F1-score for the device versus smartphone data represents a decrease in performance of less than 4 percentage points.

To evaluate whether there was a difference in overall classification errors using the device versus smartphone data, a McNemar's test was conducted, which is appropriate to use with paired nominal data representing two categories (e.g., correct versus incorrect prediction).

TABLE 3
Distribution of prediction errors between the device
and smartphone for activity recognition.

| | | Device | |
|---|---|---|---|
| | | Correct | Incorrect |
| Smartphone | Correct | 1065 | 95 |
| | Incorrect | 61 | 33 |

Finally, the Gyroscope was tested using data from the sixth activity (i.e., rotate in chair). With the Gyroscope data alone and using a rule-based model (decision tree), rotations in the chair were classified with >99% accuracy for data from both the smartphone and the device, where there are two classes (i.e., rotation versus all other activities). High levels of accuracy are possible because of the distinct 360-degree rotation at one axis of the Gyroscope in this activity.

Eight adults (50% female; Mean age=29 years, SD=13.10, Range: 18-55), including six undergraduate students who majored in theater (3 males and 3 females) and researchers who had amateur acting experiences (1 male and 1 female), participated. Participants reported on their highest level of education (50% some college, 25% bachelor's degree, 25% advanced degree) and their race and ethnicity (12.5% Black, 62.5% White non-Hispanic, 12.5% Hispanic, 12.5% more than one race).

The procedures of collecting emotional speech were partially replicated in the Ryerson Audio-Visual Database of Emotional Speech and Song (RAVDESS) corpus in a smaller scale in terms of number of participants and emotion types. RAVDESS corpus contains speech of 24 professional actors (12 female, 12 male), vocalizing two lexically matched statements, “Kids are talking by the door” and “Dogs are sitting by the door.” Eight emotional speech samples, including neutral, happy, sad, angry, fearful, surprise, and disgust expressions are recorded. Each expression is produced at two levels of emotional intensity (normal, strong).

Paralleling the validation of the motion sensor (Study 3), each participant wore a specially designed shirt that held both the device (Version 1 firmware) and a smartphone (Google Pixel, 1^{st} generation), and both the device and the smartphone were used to simultaneously record participants' speech.

To verify the quality of the emotional speech corpus, three human raters labeled each utterance using one of the above six emotion labels. Both the device and smartphone audio clips (one utterance per clip) were randomly shuffled before distributing to the human raters. Because inter-rater reliability scores fell below 0.60 for clips expressing fear, disgust, and surprise, the validation experiment was limited to 4 classes: neutral, happy, sad, and angry. This dataset includes 141 samples (neutral: 28, happy: 37, sad: 38, angry: 38) for both the device and smartphone.

In some examples, acoustic algorithms and pre-trained models may use audio at 16 kHz, wherein the collected 22 kHz samples were down sampled to 16 kHz. In instances of high frequency audio clipping, the audio stream was further processed using the built-in clipfix function of Audacity® software that finds the clipped regions of the device audio and performs interpolation of the lost signals for declipping. The threshold of clipping was empirically set to 70% without reducing amplitude for restored peaks to obtain superior audio quality. Using Version 1 of the firmware, the average proportion of missing samples, computed as a function of predicted samples based on the UTC timestamps, was 0.087 (SD=0.153).

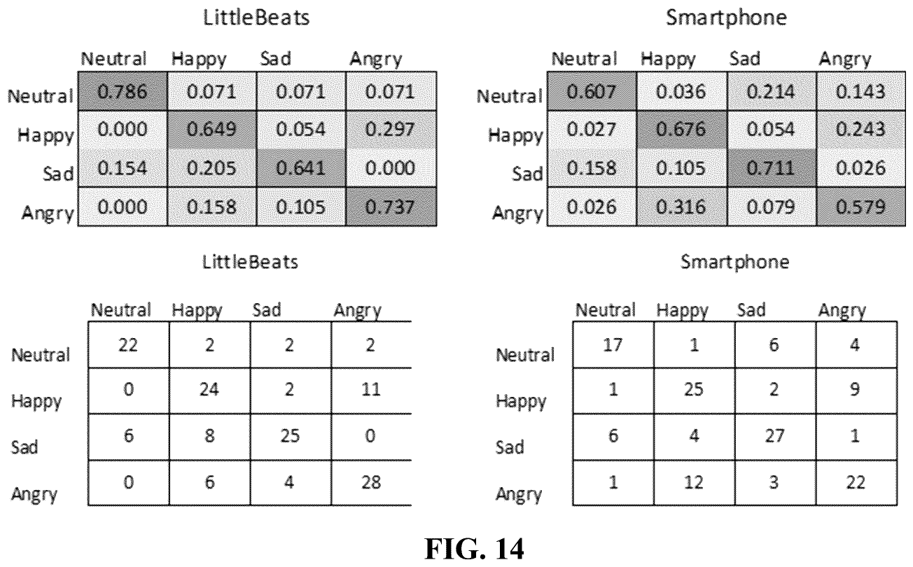

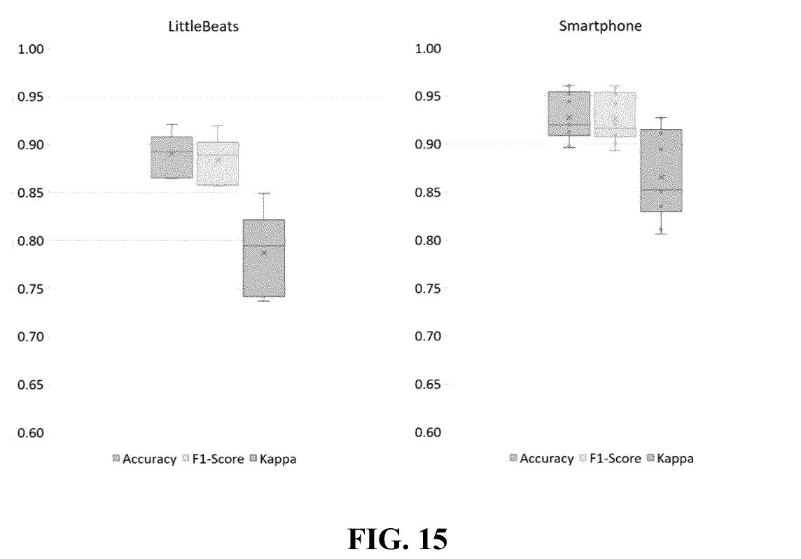

Given a relatively small corpus, sklearn package was used to implement linear discriminant analysis (LDA) for the SER validation task. The corpus was randomly split the 3 folds and perform 3-fold cross-validation tests. FIG. 14 shows the confusion matrices for the four-way emotional speech classification separately for the device and smartphone data, and FIG. 15 shows the related performance metrics (i.e., accuracy, F1-score, Cohen's kappa scores). As seen in the figures, performance on speech emotion recognition tended to be higher for data collected with the device versus smartphone.

Next, a matched-pairs test was conducted to assess whether performance on this speech emotion recognition task differed between the two recording devices (see Table 4).

TABLE 4
Distribution of prediction errors between the device
and smartphone for speech emotion recognition.

| | | Device | |
|---|---|---|---|
| | | Correct | Incorrect |
| Smart | Correct | 75 | 16 |
| phone | | | |
| | Incorrect | 23 | 27 |

Twelve adults (58.3% female; Mean age=21.74 years, SD=3.18, Range=18-26) were recruited via a university listserv and posted flyers. Participants reported on their highest level of education (8.3% high school graduate, 58.3% some college, 8.3% associate's degree, 25% bachelor's degree) and their race and ethnicity (16.7% Asian, 16.7 Black, 66.7% White non-Hispanic).

Paralleling procedures in Studies 3 and 4 above, participants wore the device (Version 2 firmware) and a Google Pixel smartphone in a t-shirt with dual pockets. While seated at a desk, participants read the Rainbow Passage aloud. The Rainbow Passage (330 words), which includes a variety of sounds and mouth movements used in unscripted English speech, has been widely used in prior work to assess speech production and reading fluency

Ground-truth transcripts were prepared using the smartphone audio passages. Annotators manually added repeated words or deleted omitted words if a participant did not read the Rainbow Passage verbatim. A bigram language model was pretrained for the Rainbow passage using KenLM software. Both CTC greedy decoding and beam search decoding was performed with beam size 25 and the language model weight was set to either 0.0 (no language model) or 2.0 (language model included with a large weight).

Table 6 below shows the WER for the device and smartphone audio with and without the language model. WER is measured by the edit distance between the reference transcripts and hypothesis transcripts generated by the ASR system. WER can be computed using the following formula,

$$W E R = \frac{S + D + I}{N} ,$$

where S, D, and I are the number of substitution errors, deletion errors, and insertion errors respectively, and N is the total number of referenced words.

TABLE 5
Word error rates (WER) for the device and smartphone
audio with and without language model.

| Models | Device WER | Smartphone WER |
|---|---|---|
| Greedy decoding | 5.75% | 3.58% |
| Beam search | 5.80% | 3.63% |
| Beam search + language | 4.16% | 2.73% |
| model | | |

Studying infants and young children in their natural environments without researchers present poses unique challenges. Unlike research with adults, commercially available wearables (e.g., FitBit, Apple watch, chest strap heart-rate monitors) are not feasible for use with infants. A compact, lightweight device was developed that captures key physiological and behavioral signals unobtrusively in the home and without researchers present. Provided herein is a comparison of performance of each device sensor against other devices that have been used extensively in the prior literature and permit a comparable form factor. Due to feasibility issues of conducting technical validations of the IMU and audio data under controlled conditions with infants and young children, validations were conducted with adult participants only for these modalities using controlled laboratory tasks prevalent in prior work.

Differences in performance between the adult and infant samples could be due to age, in that adults are more mobile than young infants and their data may be more susceptible to movement artifacts. The ECG was monitored when both adults and infants were seated. A more likely explanation is that data were collected on different versions of the device firmware, and the presence of missing samples in the ECG data only occurred in Study 1 (Version 1 firmware). Missing samples were corrected via a custom filtering/editing script, although pockets of misalignment of the device and BIOPAC IBI data were more frequent in these data. Such misalignment may result in higher error rates in the IBI data collected in Study 1 compared with Study 2, both studies were underscored showing patterns of RSA change across challenge versus baseline sessions. Taken together, this pattern of results suggests that modest level of disagreement in IBI data (likely due to some misalignment) for the adult sample did not impact measurement of cardiac vagal tone via RSA. Lastly, results from the infant data collected with Version 2 of the firmware (i.e., no missing samples), although not showing absolute 1:1 agreement with BIOPAC, indicate that the device platform is a sensor for capturing IBI data for infants under 12 months of age.

Turning to validation of the IMU, although classification of four activities (i.e., upright, walk, glide, squat) among an adult sample showed higher performance using accelerometer data from the smartphone, performance on device data was also high (e.g., F1-score=88%; kappa=0.79), and the discrepancy in F1-score was less than 4 percentage points. The smartphone acceleration values go through additional filtering via the smartphone's internal software, which likely improves performance. The device does not go through such processing and, thus, performance may increase with additional postprocessing of the device data and more complex algorithms including various filtering (e.g., Butterworth, Savitzky Golay) and smoothing techniques. In summary, Study 3 results indicate that the IMU data of the device are stable and preserve similar information as an existing mobile platform, i.e., Google Pixel 1 smartphone.

1. A wearable sensor device, comprising:
a housing sized to fit within a chest pock of a subject's garment, the housing being free of affixing features;
an electrocardiogram (ECG) sensor comprising internal circuitry within the housing and a set of electrodes to directly contact the subject's chest when the housing is disposed in the subject's garment and to conduct electrical signals to the internal circuitry;
a motion sensor disposed within the housing;
an audio sensor disposed as least partially within the housing;
a wireless communication transmitter;
a processor in communication with the ECG sensor, the motion sensor, and the audio sensor; and
a memory in communication with the processor and having instructions stored thereon that, when executed, cause the processor to:
receive, via the ECG sensor, the motion sensor, and the audio sensor, ECG data, acceleration data, and audio data; and
transmit the ECG data, acceleration data, and audio data via the wireless communication transmitter.

2. The device of claim 1, wherein the affixing features comprise at least one of a fastener, a cord, a strap, a wire, or a buckle.

3. The device of claim 1, further comprising:
digitizing circuitry, the digitizing circuitry comprising a real-time clock and synchronization circuitry;
wherein digitizing circuitry synchronizes the ECG data, acceleration data, and the audio data based on a frequency of the real-time clock.

4. The device of claim 1, wherein the audio sensor is configured to capture environmental sounds.

5. The device of claim 1, wherein the instructions, when executed, further cause the processor to:
pre-process the ECG data by filtering missing interbeat interval values.

6. A system for analyzing and monitoring biobehavioral development of a child or infant, the system comprising:
a wearable device comprising:
a plurality of child biobehavioral development sensors;
a wireless communication transmitter;
a power source configured to provide power to the wearable device for a period of time; and
a processor configured to read data from the plurality of child biobehavioral development sensors and transmit the data via the wireless communication transmitter; and
a data analysis system remote from the wearable device, the data analysis system comprising a memory having instructions stored thereon that, when executed, cause the data analysis system to:
receive, via the wireless communication transmitter, data obtained from the plurality of child biobehavioral development sensors;
determine an activity associated with the data;
determine one or more physiological and behavioral indicators based on the data and the activity; and
output a health status corresponding to the physiological and behavioral indicators.

7. The system of claim 6, wherein the health status is outputted to a remote device via the wireless communication transmitter.

8. The system of claim 6, wherein the instructions, when executed, further cause the data analysis system to:
detect an emotional state or a behavioral state associated with the one or more physiological and behavioral indicators.

9. The system of claim 6, wherein the plurality of child biobehavioral development sensors comprises at least one of an electrocardiogram (ECG) sensors, a motion sensor, or an audio sensor.

10. The system of claim 9, wherein the instructions, when executed, further cause the data analysis system to:
extract, from the data, at least one of a plurality of R-R peaks, an interbeat interval, or a heart period.

11. The system of claim 6, wherein the activity is selected from a group comprising: deep sleep, REM sleep, drowsy state, quiet alert state, active alert state, and crying.

12. The system of claim 6, wherein the instructions further cause the data analysis system to apply a random forest classifier to the data to determine the activity.

13. The system of claim 8, wherein the instructions further cause the data analysis system to:
compare the data and the emotional state or the behavioral state to a plurality of corresponding clinical threshold values.

14. The system of claim 6, wherein the instructions, when executed, further cause the data analysis system to:
determine if an intervention is needed based on the health status; and
communicate an alert.

15. A method for analyzing and long-term monitoring of child biobehavioral development, the method comprising:
receiving, via a sensing unit, ECG data, acceleration data, and audio data;
extracting one or more parameters from the ECG data, the one or more parameters including a stress level;
determining an activity associated with the acceleration data;
detecting at least one of: an emotional state, a behavioral state, or an infant-caregiver interaction associated with the audio data;
determining one or more physiological and behavioral indicators based on the one or more parameters, the activity, and the emotional state, behavioral state, or infant-caregiver interaction; and
outputting a health status corresponding to the physiological and behavioral indicators.

16. The method of claim 15, further comprising:
identifying whether the audio data originated from a user wearing the sensing unit.

17. The method of claim 15, further comprising:
detecting, via a location module, a location associated with the received ECG data, acceleration data, and audio data; and
determining if the location corresponds to a base location of the sensing unit.

18. The method of claim 15, further comprising:
extracting longitudinal information corresponding to a development of a child wearing the sensing unit.

19. The method of claim 15, further comprising:
collecting via, the sensing unit, the ECG data, acceleration data, and audio data over a period of time,
wherein the period of time is one of: at least three hours, at least ten hours, or every day for at least a week.

20. The method of claim 15, further comprising:
labeling a plurality of data groups comprising at least some of the ECG data, acceleration data, and audio data with a corresponding action.
